(19) United States
(12) Patent Application Publication
(10) Pub. No.: US 2023/0194529 A1
(43) Pub. Date: Jun. 22, 2023

(54) Rapid Assay Methods and Kits for Detecting Neutralizing Antibody for Sars-Cov-2 Using Lateral Flow Assay and Enzyme-linked Immunosorbent Assay

(71) Applicant: MISSISSIPPI STATE UNIVERSITY, Starkville, MS (US)

(72) Inventors: Keun Seok SEO, Starkville, MS (US); Nogi PARK, Starkville, MS (US); Joo Youn PARK, Starkville, MS (US)

(73) Assignee: MISSISSIPPI STATE UNIVERSITY, Starkville, MS (US)

(21) Appl. No.: 17/999,410

(22) PCT Filed: May 18, 2021
(86) PCT No.: PCT/US2021/032963

§ 371 Date: Nov. 21, 2022

Related U.S. Application Data
(60) Provisional application No. 63/175,642, filed on Apr. 16, 2021.
(60) Provisional application No. 63/028,130, filed on May 21, 2020.

(51) Int. Cl. G01N 33/569; G01N 33/543; G01N 33/68; B82Y 5/00

(52) U.S. Cl. CPC G01N 33/56983; G01N 33/54388; G01N 33/68; B82Y 5/00; G01N 2333/165; G01N 2469/20

(57) ABSTRACT

A novel assay which can differentiate a neutralizing antibody from non-neutralizing antibody which can be easily visualized, for example, by a portable UV lamp, among other visualization techniques. This assay can produce results in about 30 minutes and can be performed by untrained individuals in a non-laboratory environment. Also described is an ELISA method for determining if a human possesses at least one type of neutralizing antibody against SARS-Cov-2.

### CROSS REFERENCE TO RELATED APPLICATIONS

This application claims priority to U.S. Provisional Application No. 63/028,130, filed May 21, 2020, and U.S. Provisional Application No. 63/175,642 filed Apr. 16, 2021, the entire disclosures of which are specifically incorporated herein by reference.

### INCORPORATION OF MATERIAL OF ASCII TEXT SEQUENCE LISTING BY REFERENCE

The sequence listing submitted herewith as a text file named “MSU-1009WO_ST25” created on May 10, 2021, which is 25 kilobytes in size, is hereby incorporated by reference in its entirety.

### BACKGROUND OF THE INVENTION

The emergence of the highly pathogenic coronavirus SARS-CoV-2 and its rapid international spread has posed a serious global public-health emergency. Similar to individuals who were infected by pathogenic SARS-CoV in 2003 and Middle East respiratory syndrome coronavirus (MERS-CoV) in 2012, patients infected by SARS-CoV-2 showed a range of symptoms including dry cough, fever, headache, dyspnoea and pneumonia with significant estimated mortality rate. Since the initial outbreak, SARS-CoV-2 has spread throughout the world.

Phylogenetic analyses of the coronavirus genomes have revealed that SARS-CoV-2 is a member of the Betacoronavirus genus, which includes SARS-CoV, MERS-CoV, bat SARS-related coronaviruses (SARSr-CoV), as well as others identified in humans and diverse animal species. Bat coronavirus RaTG13 appears to be the closest relative of the SARS-CoV-2, sharing more than 93.1% sequence identity in the spike (S) gene. SARS-CoV and other SARSr-CoVs, however, are more distinct from SARS-CoV-2 and share less than 80% sequence identity.

Coronaviruses use the homotrimeric spike glycoprotein which comprises an 51 subunit and an S2 subunit in each spike monomer on the envelope to bind to cellular receptors. Such binding triggers a cascade of events that leads to fusion between cell and viral membranes for the virus to enter the cell. Previous cryo-electron microscopy studies of the SARS-CoV spike protein and its interaction with the cell receptor human angiotensin-converting enzyme 2 (ACE2) have shown that receptor binding induces the dissociation of the S1 subunit with ACE2, prompting the S2 subunit to transit from a metastable pre-fusion state to a more-stable post-fusion state that is required for membrane fusion. Therefore, binding to the ACE2 receptor is an important initial step for SARS-CoV to enter target cells. Recent studies also highlight the important role of ACE2 in mediating entry of SARS-CoV-2 into cells. HeLa cells expressing ACE2 are susceptible to SARS-CoV-2 infection whereas those without ACE2 are not. In vitro binding measurements also showed that the receptor binding domain (RBD) of the SARS-CoV-2 S1 subunit binds to ACE2 with an affinity in the low nanomolar range, indicating that the RBD is a key functional component within the S1 subunit that is responsible for binding of SARS-CoV-2 to ACE2. For a more detailed discussion on how SARS-CoV-2 enters cells using spike protein see: Lan, J. et al., Structure of the SARS-CoV-2 spike receptor-binding domain bound to the ACE2 receptor, Nature (2020); Ortegal, J. T. et al., Role of Changes in SARS-Cov-2 spike protein in the interaction with the human ACE2 receptor: An in silico Analysis, EXCLI Journal 2020; 19:410-417—ISSN 1611-2156 Received: Feb. 25, 2020, accepted: Mar. 16, 2020, published: Mar. 18, 2020; Walls, A. C. et al., Structure, Function, and Antigenicity of the SARS-CoV-2 Spike Glycoprotein, Cell, Volume 181, Issue 2, 16 Apr. 2020, Pages 281-292.

Studies have also shown that antibodies that specifically bind to the RBD of the SARS-CoV-2 S1 subunit (hereinafter “a neutralizing antibody”) confer protection from SARS-CoV-2 infection. It is thus of great interest to determine whether a person has potentially developed immunity by acquiring the neutralizing antibody from either vaccination or natural infection. Most vaccines currently developed were targeted for the original sequence of the RBD of the SARS-CoV-2 S1 subunit. However, multiple SARS-CoV-2 variants have arisen and circulated globally. In the United Kingdom (UK) and South Africa (SA) variants, the N501Y mutation is located in the RBD for cell entry which increases binding to the angiotensin-converting enzyme 2 receptor and enables the virus to expand its host range to infect mice. Another UK variant has amino acid 69 and 70 deletion (Δ69/70) and D614G substitution Amino acids 69 and 70 are located in the N-terminal domain of the spike S1 fragment. The D614G mutation is dominant in pandemic strains around the world. The other SA variant contains triple mutation in E484K+N501Y+D614G. These amino acids are located in the viral RBD. Experimental evidence showed that the E484K substitution alone confers resistance to several neutralizing monoclonal antibodies. Thus, it is of interest whether current vaccines administered worldwide can still effectively neutralize SARS-CoV-2 variants. However, current immunological assays for detecting immunoglobulin (Ig) G or IgM but cannot distinguish whether these IgG and IgM are neutralizing antibodies to protect people from SARS-CoV-2.

The lateral flow assay (LFA) is a paper-based platform for the detection and quantification of analytes in complex mixtures, where the sample is placed on a test device and the results are displayed within 5-30 minutes. Low development costs and ease of production of LFAs have resulted in the expansion of its application to multiple fields in which rapid tests are required. LFA-based tests are widely used in hospitals, physician's offices and clinical laboratories for the qualitative and quantitative detection of specific antigens and antibodies, as well as products of gene amplification. A variety of body fluid samples can be tested using LFAs, including urine, saliva, sweat, serum, plasma, whole blood and other body fluids. For background on LFA see for example: Koczul et al., Essays in Biochemistry (2016) 60 111-120, and U.S. Pat. No. 6,485,982.

Typically, an LFA apparatus includes a sample pad, a conjugate release pad (or simply “conjugate pad”), a nitrocellulose strip that contains Positive and Negative lines, and a wicking pad. (See FIG. 1). Each component of the apparatus overlaps by at least 1-2 mm which enables unimpeded capillary flow of the sample throughout the apparatus.

To use the LFA device, a liquid sample such as whole blood, serum, plasma, urine, saliva, or solubilized solids, is added directly to the sample pad and is wicked through the LFA device. The sample pad neutralizes the sample and filters unwanted particulates such as red blood cells. The sample can then flow to the conjugate pad that contains an antibody labeled with a detectable marker such as a label or tag, for example, strongly colored or fluorescent nanoparticles. When the liquid reaches the conjugate pad, these dried nanoparticles are released and mix with the sample. If there are any target analytes in the sample that the antibody recognizes, these will bind to the antibody.

The analyte-bound nanoparticles then flow through a nitrocellulose membrane and across one or more Positive lines and a Negative line. The Positive line (the orange line in FIG. 1) is the primary read-out of the diagnostic and consists of immobilized proteins that can bind the analyte-bound nanoparticle to generate a signal that is correlated to the presence of the analyte in the sample. The fluid continues to flow across the strip until it reaches the Negative line. The Negative line (grey line in FIG. 1) contains affinity ligands that will bind the nanoparticle conjugate without the analyte present in solution to confirm that the assay is working properly. After the Negative line, the fluid flows into the absorbent pad (wicking pad) which absorbs sample liquid to ensure that there is consistent flow across the Positive and Negative lines. In some tests, a chase buffer is applied to the sample port after sample introduction to ensure that all of the sample is transported across the strip Once all the sample has passed across the Positive and Negative lines, the assay is complete and the user can read the results.

Traditionally LFA employs two common assay formats, “sandwich” and “competitive”. The sandwich assay format is typically used for detecting larger analytes that have at least two binding sites, or epitopes. Usually, an antibody to one binding site is conjugated to the nanoparticle, and an antibody to another binding site is used for the assay's Positive line. If there is analyte present in the sample, the analyte will bind to both the antibody-nanoparticle conjugate and to the antibody on the Positive line, yielding a positive signal. The sandwich format results in a signal intensity at the Positive line that is directly proportional to the amount of analyte present in the sample. Regardless of the quantity of analyte in the sample, an anti-species antibody at the Negative line will bind the nanoparticle, yielding a strong Negative line signal that demonstrates that the assay is functioning correctly. Exemplary analytes detected by the sandwich assay include the p24 antigen in the HIV test and human chorionic gonadotropin (hCG) in the pregnancy test.

The competitive format is used for detecting analytes when antibody pairs are unavailable or if the analyte is too small for multiple antibody binding events, such as steroids and drugs. In this format, the Positive line typically contains the analyte molecule, usually a protein-analyte complex, and the conjugate pad contains the detection antibody-nanoparticle conjugate. If the target analyte is present, the analyte will bind to the conjugate and prevent it from binding to the analyte at the Positive line. If the analyte is not present, the conjugates will bind to the analyte at the Positive line, yielding a signal. In the competitive format, the signal intensity is inversely proportional to the amount of analyte present in the sample. As in the sandwich format, the Negative line will bind the nanoparticle conjugate with or without the analyte, providing confidence that the assay is working correctly.

The enzyme-linked immunosorbent assay (ELISA) is a common analytical biochemistry assay that can be used as a diagnostic tool in medicine. It uses a surface coated with an antigen of interest which is captured by antigen specific antibody conjugated with enzyme such as alkaline phosphatase or horse radish peroxidase (HRP). A binding of antibody to target antigen is visualized by adding enzyme's substrate which subsequently produces a detectable signal, most commonly a color change.

The typical chemical conjugation of enzyme or other visual signal molecules involves oxidization of polysaccharide residues in the enzyme with sodium periodate which convert polysaccharide to reactive aldehyde groups that can conjugate with amino groups of target protein and produce Schiff bases. Although it is highly effective, this method can conjugate different numbers of enzyme or visual signal molecules to target protein since target protein typically possesses multiple amino groups. This heterogenous conjugate (i.e. the numbers and locations of conjugate are heterogeneous) can generate inconsistency in the sensitivity and specificity of conjugated protein. It also demands to use a large amount of conjugated protein since only the fraction of conjugated protein is able to interact with its ligand. To overcome the drawbacks of chemical conjugation method, we generate chimeric proteins in which the gene encoding the RBD of the SARS-CoV-2 S1 subunit was in-frame fused with the gene encoding horse radish peroxidase. These chimeric proteins homogenously interact with a target protein which significantly increase sensitivity and specificity of assay.

The present invention relates to assays (kits and methods) which employ novel lateral flow assay and ELISA formats. The kits and methods can differentiate between neutralizing antibody to SARS-Cov-2 and non-neutralizing antibody thereby allowing a determination if a person possesses neutralizing antibody to SARS-Cov-2. The assays allow easy visualization, for example, by using a portable UV lamp or horseradish peroxidase activity, among other possible visualization techniques. These assays also can produce results in about 30 minutes or less and can be performed by untrained individuals without requiring a specialized environment.

### SUMMARY OF THE INVENTION

In one embodiment, the present invention relates to the generation of wild type, UK variant, and SA variant of RBD of SARS-CoV-2 fused with horse radish peroxidase expressed in human HEK293t cell line.

In another embodiment, the present invention relates to the generation of ACE2 fused with mouse immunoglobulin G1 fragment expressed in human HEK293t cell line.

In another embodiment, the present invention relates to a method for determining if a human possesses at least one type of neutralizing antibody against SARS-Cov-2. The method comprises steps of:
a) incubating a body fluid with Chimeric Protein 1 for a period of time to form Mixture 1 (SARS-CoV-2-RBD-HRP and neutralizing antibody complex);
b) adding Chimeric Protein 2 to Mixture 1 formed in step (a) and incubating for a period of time to form Mixture 2;
c) contacting Mixture 2 with anti-hIgG;
d) contacting Mixture 2 with anti-mIgG;
e) visualizing whether the anti-hIgG or the anti-mIgG produces a visible signal emitted by tag contained within Chimeric Protein 1;
f) concluding the body fluid contains at least one type of neutralizing antibody against SARS-Cov-2 when the visible signal is detected from the anti-hIgG, or concluding that the body fluid does not contain at least one type of neutralizing antibody against SARS-Cov-2 when the visible signal is detected from the anti-mIgG.

The foregoing method may also include further steps of adding rabbit IgG conjugated to a tag to Mixture 2 prior to step (c), contacting the Mixture 2 from step (d) with anti-rIgG and determining if a visible signal emitted by the tag attached to the rabbit IgG indicates proper operation of the assay and ensures that an adequate amount of sample has been employed in the test.

In one embodiment, the method steps in the paragraphs [0020]-[0023] above are conducted sequentially from (a) to (f).

In another embodiment, the method steps in the paragraphs [0020]-[0024] above, steps (c) and (d) are conducted sequentially with step (c) being performed prior to step (d).

In another embodiment, the method steps in the paragraphs [0020]-[0025] above, steps (c) and (d) are conducted simultaneously.

The anti-hIgG is an anti-human IgG that functions as a positive indicator showing the presence of neutralizing antibody. The anti-mIgG is an anti-mouse IgG1 that functions as a negative indicator showing the absence of neutralizing antibody. The optional anti-rIgG is an anti-rabbit IgG that is used as a control to show the presence of a sufficient amount of an appropriate sample.

In a specific embodiment, the above method for determining if a human possesses at least one type of neutralizing antibody against SARS-Cov-2, is a lateral flow method. The lateral flow method comprises steps of:
a) incubating a body fluid with Chimeric Protein 1, Chimeric Protein 2, or Chimeric Protein 3 for a period of time to form Mixture 1 (SARS-CoV-2-RBD-HRP and neutralizing antibody complex);
b) adding Chimeric Protein 4 to Mixture 1 formed in step (a) and incubating for a period of time to form Mixture 2 (SARS-CoV-2-RBD-HRP and ACE2-mIgG1Fc complex);
c) adding rabbit IgG conjugate with nanogold particles (rIgG-AuNP) to indicate that the assay is working properly and that a sufficient amount of sample has been employed;
d) adding Mixture 1 and Mixture 2 to a unit comprising the following elements laid out in the following successive order:
   1. a sample pad onto which Mixture 1 and Mixture 2 is loaded;
   2. a test pad in contact with the sample pad including a positive line in which anti-hIgG is immobilized, a negative line in which anti-mIgG is immobilized downstream from the positive line, and, optionally, a control line in which the anti-rIgG is immobilized downstream from the negative line;
   3. an optional absorption pad in contact with the test pad which facilitates the flow of Mixture 2 from the sample pad through the test pad; and
   4. an optional backing card onto which the sample pad, test pad and optional absorption pad is mounted.
e) contacting Mixture 2 with anti-hIgG (anti-human IgG) that functions as a positive indicator showing the presence of neutralizing antibody;
f) contacting Mixture 2 with anti-mIgG (anti-mouse IgG1) that functions as a negative indicator showing the absence of neutralizing antibody;
g) contacting Rabbit IgG-AuNP with anti-rIgG (anti-rabbit IgG) that is used as a control to show the presence of a sufficient amount of an appropriate sample;
h) visualizing whether the anti-hIgG or the anti-mIgG produces a visible signal emitted by horse radish peroxidase activity contained within Chimeric Protein 1, 2, or 3, when a substrate (ADHP, 10-Acetyl-3,7-dihyroxyphenoxazine is loaded on to the sample pad; and
i) concluding that the body fluid contains at least one type of neutralizing antibody against SARS-Cov-2 when the visible signal is detected from the anti-hIgG, or concluding that the body fluid does not contain at least one type of neutralizing antibody against SARS-Cov-2 when the visible signal is detected from the anti-mIgG;
j) determining that the anti-rIgG indicates a sufficient amount of sample has been employed by the presence of a visually detectable signal; and
wherein optionally, the body fluid in step (a) is whole blood or serum; the period of time in step (a) is 10-15 minutes, the period of time in step (b) is 10-15 minutes, and/or Chimeric protein 1, 2, and 3 each have oxidase activity.

In another specific embodiment, the above method for determining if a human possesses at least one type of neutralizing antibody against SARS-Cov-2, is carried out using an ELISA method.

The ELISA method comprising steps of:
a) incubating a body fluid with Chimeric Protein 1, 2, or 3 for a period of time to form SARS-CoV-2-RBD-HRP and a neutralizing antibody complex or unbound free SARS-CoV-2-RBD-HRP;
b) adding mixture in a) to a 96-well plate coated with Chimeric protein 4; and
c) capturing unbound Chimeric Protein 1, 2, or 3 on the 96-well plate, while Chimeric Protein 1, 2, or 3 that is bound to the neutralizing antibody is removed during washing with phosphate buffer;
d) visualizing horse peroxidase activity within Chimeric Protein 1, 2, or 3 is by adding a substrate (ADHP, 10-Acetyl-3,7-dihyroxyphenoxazine);
e) concluding that the body fluid contains at least one type of neutralizing antibody against SARS-Cov-2 when the a signal is not visible, since the interaction of Chimeric Protein 1, 2, or 3 and Chimeric protein 4 is inhibited by the body fluid;
f) concluding that the body fluid does not contain at least one type of neutralizing antibody against SARS-Cov-2 when the signal is visible, since the interaction of Chimeric Protein 1, 2, or 3, and Chimeric protein 4 is not inhibited by the body fluid; and wherein Chimeric Protein 1, 2, or 3 is wild type, UK variant, or SA variant of RBD of SARS-CoV-2 fused with horse radish peroxidase; and Chimeric Protein 4 is ACE2 fused with mouse immunoglobulin G1 fragment.

In one preferred embodiment, in the above methods of paragraphs [0028]400291, the body fluid is whole blood or serum; the period of time in step (a) is 10-15 minutes; and the period of time in step (b) is 10-15 minutes.

In another embodiment, the present invention may be a kit for determining if a human has neutralizing antibodies against SARS-Cov-2, the kit comprising:
a) a container containing Chimeric Protein 1 into which a body fluid may be added to form Mixture 1;
b) Chimeric Protein 2 to be added to Mixture 1 to form Mixture 2;
c) a unit comprising the following elements laid out in the following successive order:
   1. a sample pad onto which Mixture 2 is loaded;
   2. a test pad in contact with the sample pad including a positive line in which anti-hIgG is immobilized, a negative line in which anti-mIgG is immobilized downstream from the positive line, and, optionally, a control line in which the anti-rIgG is immobilized downstream from the negative line;
   3. an optional absorption pad in contact with the test pad which facilitates the flow of Mixture 2 from the sample pad through the test pad; and
      an optional backing card onto which the sample pad, test pad and optional absorption pad is mounted.

The kits of the present invention may further include instructions for use of the kit for determining if a human has at least one type of neutralizing antibody against SARS-Cov-2.

### BRIEF DESCRIPTION OF THE DRAWINGS

FIG. 1 shows a traditional lateral flow assay kit.

FIG. 2A depicts the reagents, Chimeric Protein 1 and Chimeric Protein 2, used in the present invention, and also depicts the reaction of Protein 1 with Chimeric Protein 2 and the neutralizing antibody to SARS-Cov-2.

FIG. 2B depicts the reagents, Chimeric Protein 1 and Chimeric Protein 2, Chimeric Protein 3, and Chimeric Protein 4, used in the present invention, and also depicts the reaction of Chimeric Protein 1, 2, or 3 with Chimeric Protein 4 and the neutralizing antibody to SARS-Cov-2.

FIGS. 3A and 3B each shows an embodiment of a lateral flow assay kit of the present invention and describes how to determine the results of the assay.

FIG. 4 shows an embodiment of an ELISA method of the present invention and describes how to determine the results of the assay.

FIG. 5 shows the amino acid sequence of the original RBD of the SARS-Cov-2 spike protein (Chimeric Protein 1, SEQ ID NO: 1). Bold indicates the original RBD of the spike protein portion.

FIG. 6 shows the amino acid sequence of the UK variant RBD of the SARS-Cov-2 spike protein (Chimeric Protein 2, SEQ ID NO: 2). Bold indicates the UK variant RBD of the spike protein portion.

FIG. 7 shows the amino acid sequence of the SA variant RBD of the SARS-Cov-2 spike protein (Chimeric Protein 3, SEQ ID NO: 3). Bold indicates the SA variant RBD of the spike protein portion.

FIG. 8 shows the amino acid sequence of the ACE2-mIgG1Fc protein (Chimeric Protein 4, SEQ ID NO: 4). Bold indicates the ACE receptor protein portion.

FIG. 9 shows the amino acid sequence of the rabbit IgG (SEQ ID NO: 5).

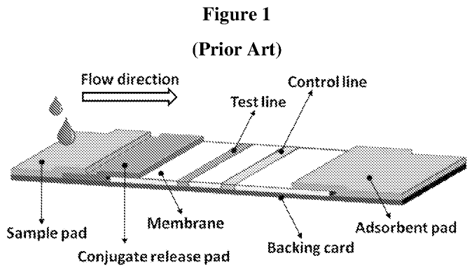

### DETAILED DESCRIPTION OF THE INVENTION

In one aspect, the present invention provides a method for rapid detection of neutralizing antibody against SARS-CoV-2, such as neutralizing IgG antibody against SARS-CoV-2. SARS-CoV-2 expresses a unique spike protein that interacts with human angiotensin-converting enzyme 2 (ACE2) for invading human respiratory epithelial cells. Studies have shown that the neutralizing IgG antibody that binds to the receptor binding domain (RBD) of the spike protein that interacts with ACE2 is capable of reducing or neutralizing the virulence of SARS-CoV-2. Currently, multiple variants of SARS-CoV-2 are circulating globally. The United Kingdom (UK) identified a variant called B.1.1.7 carrying a mutation in the spike protein (N501Y) that affects the conformation of RBD. In South Africa (SA), another variant called B.1.351 sharing mutations in B.1.1.7 as well as additional mutation in the spike protein (E484K) that can provide resistance to current Covid-19 vaccine.

In order to detect the neutralizing antibody that binds to the RBD of the spike protein that inhibit interaction of spike protein with human ACE2, we generated Chimeric proteins in which the variants of RBD spike protein is fused with horse radish peroxidase (HRP). For example, Chimeric Protein 1 (SARS-CoV-2-RBD-GFPuv) is used to bind to this receptor binding domain (RBD) of the spike protein. Chimeric protein 1 includes a marker such as a fused green fluorescence protein (GFPuv) for the visualization of the assay result.

Chimeric protein 1 includes an original RBD fused with the HRP. Chimeric protein 2 includes the UK variant RBD fused with the HRP, Chimeric protein 3 includes the SA variant RBD fused with the HRP. We generated another Chimeric Protein 3 (ACE2-mIgG1Fc) in which the extracellular domain of the ACE2 is fused with constant regions of mouse immunoglobulin G1 (IgG).

In one embodiment, the method of the invention is carried out using a lateral flow assay format. In lateral flow assay, body fluids will be pre-incubated the RBD-HRP chimeric proteins, followed by the ACE2-mIgG1Fc. In the presence of neutralizing antibody, the neutralizing antibody will bind to the RBD-HRP, so the entire neutralizing antigen-antibody complex will be captured at the anti-hIgG line (Positive line). In the absence of neutralizing antibody, the RBD-HRP will bind to the ACE2-mIgG1Fc, so the entire protein-protein complex will be captured at the anti-mIgG line (negative line). The horse radish peroxidase activity within RBD-HRP chimeric proteins will be visualized by adding a substrate (ADHP, 10-Acetyl-3,7-dihyroxyphenoxazine) which generate visible colorimetric signal as well as fluorescent signal at 570 nm.

The amount of RBD-HRP chimeric proteins captured by neutralizing antibody may be empirically determined and compared to a standard to identify whether the neutralizing antibody is present at protective levels.

For instance, Chimeric protein 1 will bind to the neutralizing IgG antibody, so that the entire antigen-antibody complex will bind to the anti-hIgG (Positive line). The amount of Chimeric protein 1 captured by neutralizing antibody may be empirically determined and compared to a standard to identify whether the neutralizing antibody is present at protective levels.

Similarly, as another example, Chimeric Protein 2 (ACE2-mIgG1Fc) in which the extracellular domain of ACE2 is fused with constant regions of mouse immunoglobulin G1 (IgG) that will bind to anti-mIgG (negative line) for detection of SARS-CoV-2-RBD-GFPuv complex in human IgG is also used in the assay as an indicator of the absence or substantial absence of the neutralizing antibodies.

To be sure that the assay is working properly, rabbit IgG conjugate with nanogold particles (AuNP). The rabbit IgG conjugate will bind to the anti-rabbit IgG (control line) to indicate that the assay is working properly and that a sufficient amount of sample has been employed.

In the other embodiment, the method of the invention is carried out using an ELISA format. In ELISA, body fluids will be pre-incubated the RBD-HRP chimeric proteins to allow the binding of neutralizing antibody to the RBD-HRP. The mixture will be then added to the capture plate on which is pre-coated with the the ACE2-mIgG1Fc. The unbound RBD-HRP will be captured on the plate, while the neutralizing antibody and the RBD-HRP chimeric protein complex will be removed during washing. The horse radish peroxidase activity within RBD-HRP chimeric proteins will be visualized by adding a substrate (ADHP, 10-Acetyl-3,7-dihyroxyphenoxazine) which generate visible colorimetric signal as well as fluorescent signal at 570 nm.

In one embodiment, the method is conducted using Positive and Negative lines specifically designed to determine if the sample contains a protective level of neutralizing antibody. In this embodiment, when the sample contains a protective level of neutralizing antibody, there will be a significantly stronger visible signal at the Positive line. If the sample does not contain any neutralizing antibody or only a small amount of neutralizing antibody, there will be a significantly stronger visible signal at Negative line. When the sample contains some neutralizing antibody but less than the protective level of neutralizing antibody, there will be strong visible signals at both of the Positive and Negative lines.

In another aspect, the present invention relates to a kit for carrying out the above described methods of the present invention.

### Specific Embodiments

If a protective level of neutralizing antibody is present in the sample of body fluid, SARS-CoV-2-RBD-HRP chimeric proteins (Chimeric protein 1, 2, and 3) will substantially bind or completely bind with the neutralizing human IgG antibody. This will prevent subsequent binding of ACE2-mIgG1Fc to SARS-CoV-2-RBD-HRP.

If a protective level of neutralizing antibody is not present in the sample of body fluid, the first incubation of SARS-CoV-2-RBD-HRP chimeric proteins (Chimeric protein 1, 2, and 3) with the body fluid sample will leave some free SARS-CoV-2-RBD-HRP chimeric proteins. This will allow the downstream ACE2-mIgG1Fc (Chimeric Protein 4) to bind to SARS-CoV-2-RBD-HRP chimeric proteins, resulting in a complex of the SARS-CoV-2-RBD-HRP chimeric proteins and ACE2-mIgG1Fc.

If a protective level of neutralizing antibody is present in the sample of body fluid, SARS-CoV-2-RBD-GFPuv (Chimeric Protein 1) will substantially completely bind or completely bind with the neutralizing human IgG antibody. This will prevent subsequent binding of ACE2-mIgG1Fc to SARS-CoV-2-RBD-GFPuv.

If a protective level of neutralizing antibody is not present in the sample of body fluid, the first incubation of SARS-CoV-2-RBD-GFPuv (Chimeric Protein 1) with the body fluid sample will leave some free SARS-CoV-2-RBD-GFPuv (Chimeric Protein 1). This will allow the downstream ACE2-mIgG1Fc (Chimeric Protein 2) to bind to SARS-CoV-2-RBD-GFPuv, resulting in a complex of the SARS-CoV-2-RBD-GFPuv and ACE2-mIgG1Fc.

In one specific embodiment, the novel lateral flow assay kit of the invention may contain the anti-human IgG antibody immobilized (printed) at the Positive line of the assay device and the anti-mouse IgG antibody immobilized (printed) at the Negative line of the assay device. The complex of SARS-CoV-2-RBD-HRP chimeric proteins and human neutralizing IgG will be captured at the Positive line formed by the anti-human IgG antibody. The complex of SARS-CoV-2-RBD-HRP chimeric proteins and ACE2-mIgG1Fc will be captured at the Negative line formed by the anti-mouse IgG antibody. The horse radish peroxidase activity signal produced by the substrate (ADHP) can be visualized as a colorimetric signal or fluorescent signal.

In another specific embodiment, the novel lateral flow assay kit of the invention may contain the anti-human IgG antibody immobilized (printed) at the Positive line of the assay device and the anti-mouse IgG antibody immobilized (printed) at the Negative line of the assay device. The complex of SARS-CoV-2-RBD-GFPuv and human IgG will be captured at the Positive line formed by the anti-human IgG antibody. The complex of SARS-CoV RBD-GFPuv and ACE2-mIgG1Fc will be captured at the Negative line formed by the anti-mouse IgG antibody. The GFPuv signal produced by the fluorescent marker can be visualized using a UV or wood lamp.

If samples contain protective levels of neutralizing antibody, there will be a visible signal only at the Positive line. If samples do not contain any neutralizing antibody, there will be visible signal only at Negative line. If samples contain some neutralizing antibody but not a protective level, there will be visible signals at both Positive and Negative lines. When test results show a visible signal only at the Positive line, this corresponds to protection from SARS-COV-2.

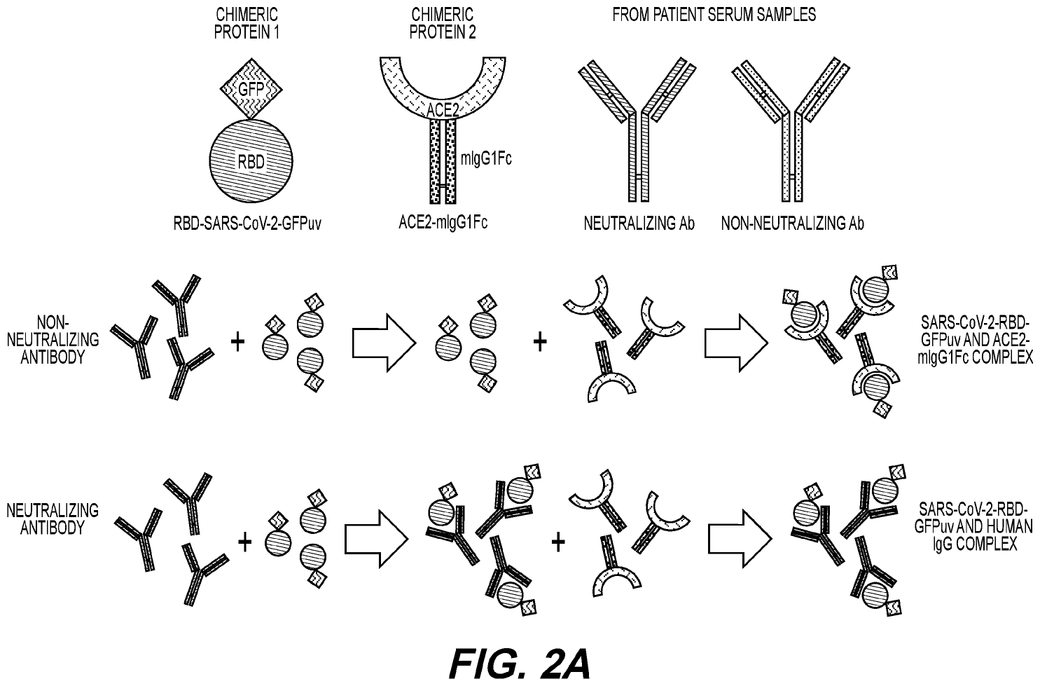

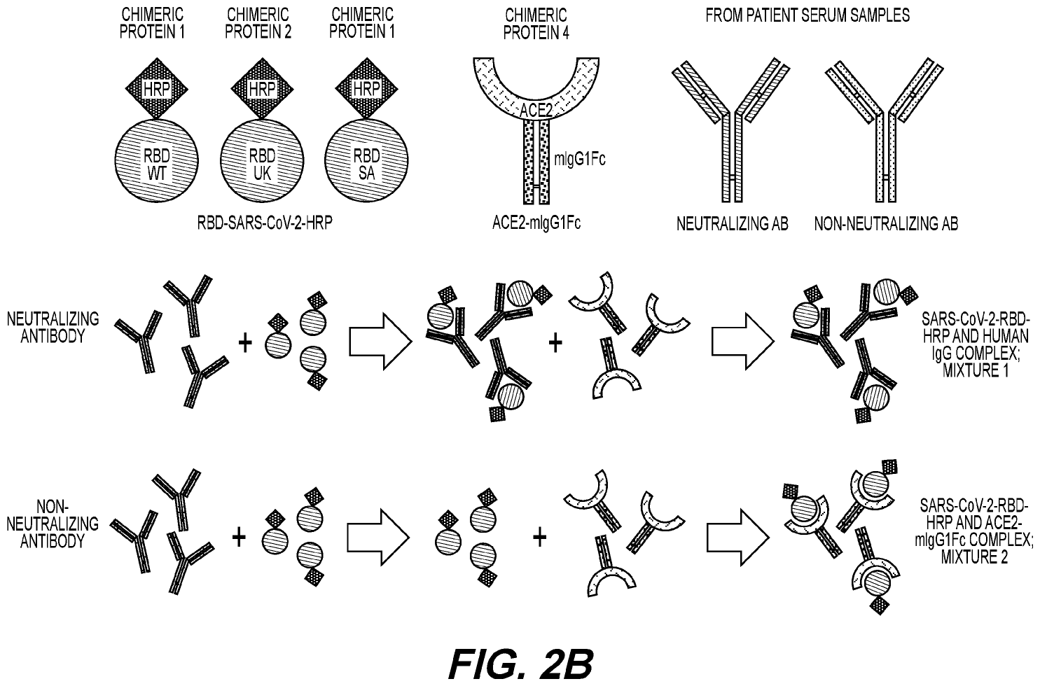

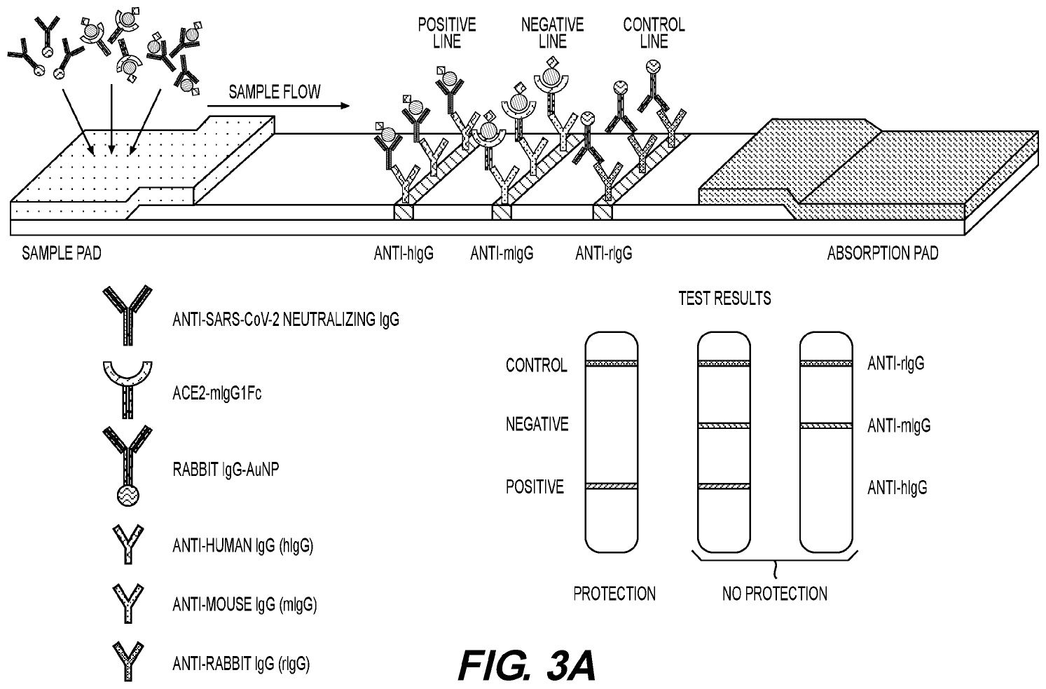

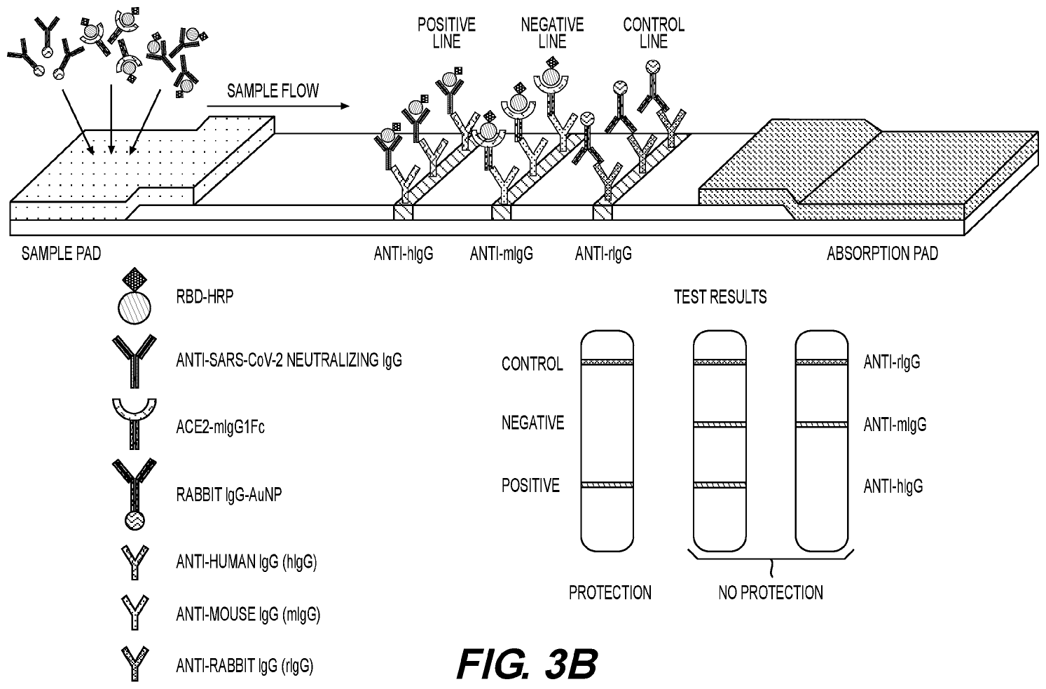

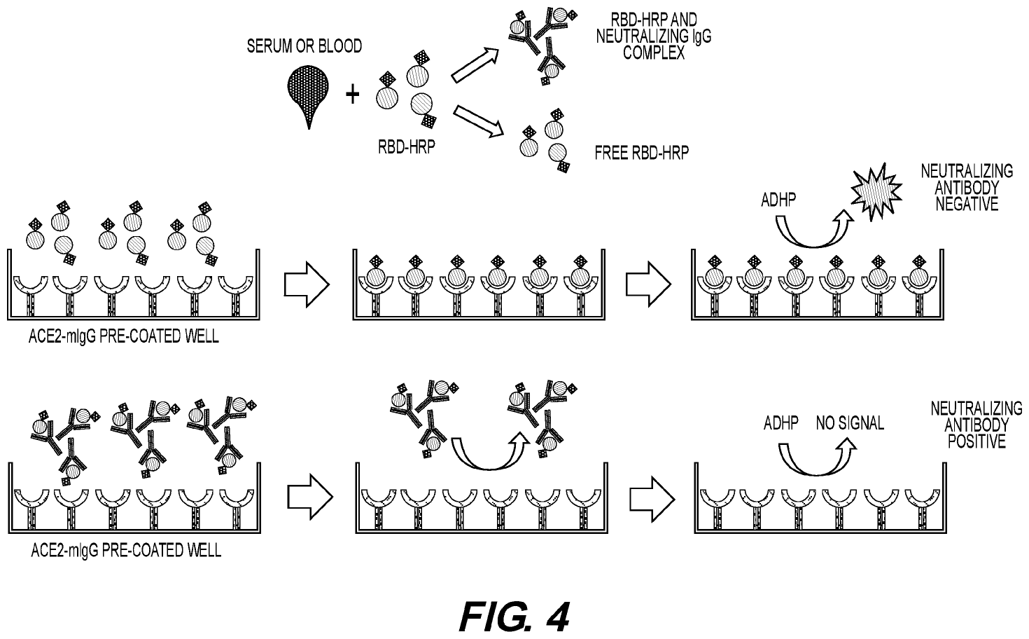

Schematic illustrations of the assay are shown in FIGS. 2A-2B and 3A-3B.

“Neutralizing antibody” as used herein refers to an antibody that specifically binds to the RBD of the spike protein of SARS-COV-2, and defends a cell from SARS-COV-2 by neutralizing the ability of the RBD of the spike protein of SARS-COV-2 to bind to the ACE2 receptor. Neutralization renders SARS-COV-2 non-infectious and non-pathogenic. Neutralizing antibodies are part of the humoral response of the immune system against the virus. Neutralizing antibodies prevent the virus particle from interacting with the ACE2 receptor of host cells thereby preventing infection of the host cells.

“Body fluids” are liquids originating from inside the body of a living human. Body fluids include fluids that are excreted or secreted from the body. Exemplary body fluids include:
Aqueous humour and vitreous humour
Bile
Blood serum or whole blood
Breast milk
Cerebrospinal fluid
Cerumen (earwax)
Endolymph and perilymph
Female ejaculate
Gastric juice
Mucus (including nasal drainage and phlegm)
Peritoneal fluid
Pleural fluid
Saliva
Sebum (skin oil)
Semen
Sweat
Tears
Vaginal secretion
Vomit
Urine

Any of the foregoing body fluids can be tested using the assay apparatus of the present invention. However, in most cases the tested body fluid is typically whole blood, saliva, or blood serum.

The novel LFA apparatus of the present invention comprises the following elements set forth in successive order (see FIGS. 3A-3B):
a. a sample pad;
b. a test pad including a Positive line containing immobilized anti-hIgG, a downstream negative line containing immobilized anti-mIgG and an optional control line downstream from the negative line containing immobilized anti-rIgG;
c. an optional absorption pad to facilitate the flow of Mixture 2 from the sample pad through the test pad; and
d. an optional backing card to position and hold elements a-c above.

The sample pad and test pad can be made from a permeable material, e.g., nitrocellulose, glass fiber, capable of transporting an aqueous solution by capillary action, wicking, or simple wetting. The backing card (the black element in FIGS. 3A-3B) can be made from inert hard material such as polyvinyl chloride, polypropylene, or other thermoplastic resins.

Exemplary LFA Protocol

A body fluid is mixed with the RBD-HRP chimeric proteins (Chimeric Protein 1, 2, and/or 3) in a container (e.g. a plastic well) and incubated for about 10 to 15 minutes to form Mixture 1. Subsequently, Chimeric Protein 4 is added to Mixture 1 and incubated for an additional 10 to 15 minutes to form Mixture 2.

Mixture 2 and rabbit IgG conjugated with AuNP are applied to the sample pad shown in FIG. 3B. Mixture 2 will be transported downstream by capillary action, wicking, or simple wetting from the sample pad to the test pad (white portion in FIG. 3B) where it encounters the Positive line and the Negative line and the sample is transported further until it encounters the Control line.

An optional absorption pad can be located downstream of the test pad and the test pad and absorption pad are provided without contacting surfaces so that Mixture 2 can flow from the test pad to the absorption pad. After Mixture 2 passes through the Negative line, it moves to the absorption pad which may be composed of sorbent or a super sorbent material. The purpose of absorption pad is to assure that Mixture 2 is drawn through the Positive and Negative lines. Thus, the absorption pad is preferably sized to ensure that all of Mixture 2 can reach at least the Negative line during the test.

The Positive and negative lines are printed with a predetermined amount of immobilized goat anti-hIgG and anti-mIgG, respectively. Any suitable conventional immobilization technique may be employed to print or apply the Positive and Negative lines.

Suitable sorbents which can be used in the absorption pad can include commercial materials of the type available, for example, from The Dow Chemical Company of Midland, Mich., and the Chemical division of American Colloid, Arlington Heights, Ill. These materials can absorb many times their weight in water and are commonly used in disposable diapers. They typically comprise lightly crosslinked polyacrylate salts, typically alkali metal salts.

The RBD-HRP chimeric protein (Chimeric protein 1, 2, and/or 3) contains a horse radish peroxidase activity which can be visualized by adding a substrate (ADHP) that generates a colorimetric signal or fluorescent signal. Alternatively, the RBD-HRP chimeric protein (Chimeric protein 1, 2, and/or 3) contains a GFPuv label which can be visualized by using a lamp or UV light. When color appears at the Positive line, the presence of neutralizing antibody is indicated, whereas when color appears at the Negative line, the absence of the neutralizing antibody is indicated. A color chart can optionally be provided for aiding in interpretation of the results of the assay.

The label, tag or marker used in the RBD-HRP chimeric proteins may be, for example, horse peroxidase activity (HRP) or Green Fluorescent Protein UV variant (GFPuv). However, other labels such metal sol, such as colloidal gold, and other types of colored or fluorescent particles known to be useful as marker substances in immunoassay procedures can also be used instead of the HRP or GFPuv. See, for example, U.S. Pat. No. 4,313,734, Feb. 2, 1982, to Leuvering, the disclosure of which is incorporated herein by reference. For details and engineering principles involved in the synthesis of colored particle conjugates see Horisberger, Evaluation of Colloidal Gold as a Cytochromic Marker for Transmission and scanning Electron Microscopy, Biol. Cellulaire, 36, 253-258 (1979); Leuvering et al, Sol Particle Immunoassay, J Immunoassay 1 (1), 77-91 (1980), and Frens, Controlled Nucleation for the Regulation of the Particle Size in Monodisperse Gold Suspensions, Nature, Physical Science, 241, pp. 20-22 (1973).

Other assay methods besides LFA methods can also be used to carry out the method of the present invention, including, for example ELISA assays.

### Example 1 Making the Chimeric Protein 1

The original receptor binding domain of the SARS-CoV-2 spike protein gene was amplified by polymerase chain reaction from the plasmid obtained from the University of Georgia. This source plasmid is also available from the Biodefense and Emerging Infections Research Resources Repository (BEI Resources).

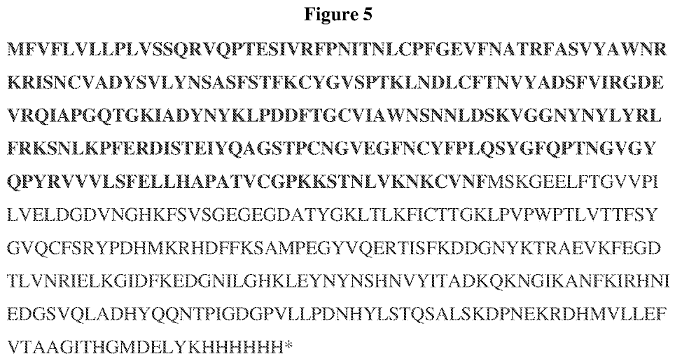

To express the horse radish peroxidase (HRP) in the human cell line, the codon usage of the gene encoding horseradish peroxidase (GenBank: M3715) was optimized by the codon usage frequency table for human (Athey, J., Alexaki, A., Osipova, E. et al. A new and updated resource for codon usage tables. BMC Bioinformatics 18, 391 (2017) (doi.org/10.1186/s12859-017-1793-7). The codon optimized HRP gene was synthesized (Blue Heron) and amplified by polymerase chain reaction. Two gene fragments were joined by overlap extension polymerase chain reaction and cloned into pLenti6 V5 expression plasmid (Invitrogen) for expression in HEK293T cells. The sequence of Chimeric Protein 1 is shown in FIG. 5. Bold indicates the original RBD of the spike protein portion.

### Example 2 Making the Chimeric Protein 2

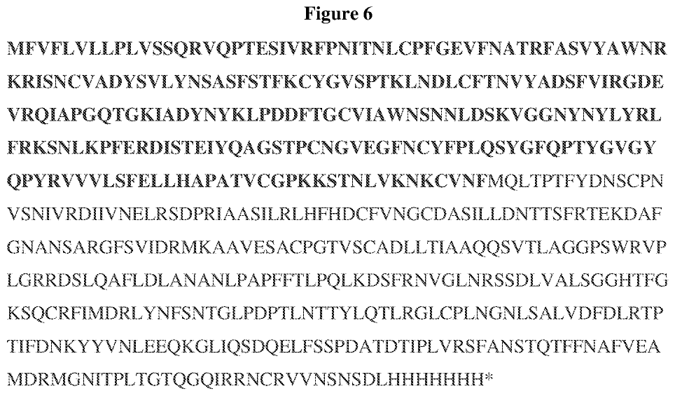

The gene encoding the UK variant RBD of spike protein was synthesized (Blue Heron) and amplified by polymerase chain reaction. This gene fragment was in-frame fused with the codon optimized horse radish peroxidase gene fragment by overlap extension polymerase chain reaction and cloned into pLenti6 V5 expression plasmid (Invitrogen) for expression in HEK293T cells. The sequence of Chimeric Protein 2 is shown in FIG. 6. Bold indicates the UK variant RBD of the spike protein portion.

### Example 3 Making the Chimeric Protein 3

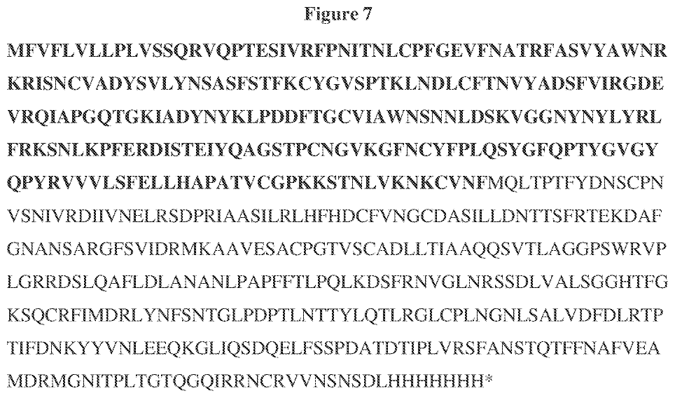

The gene encoding the SA variant RBD of spike protein was synthesized (Blue Heron) and amplified by polymerase chain reaction. This gene fragment was in-frame fused with the codon optimized horse radish peroxidase gene fragment by overlap extension polymerase chain reaction and cloned into pLenti6 V5 expression plasmid (Invitrogen) for expression in HEK293T cells. The sequence of Chimeric Protein 3 is shown in FIG. 7. Bold indicates the SA variant RBD of the spike protein portion.

### Example 4 Making of Chimeric Protein 4

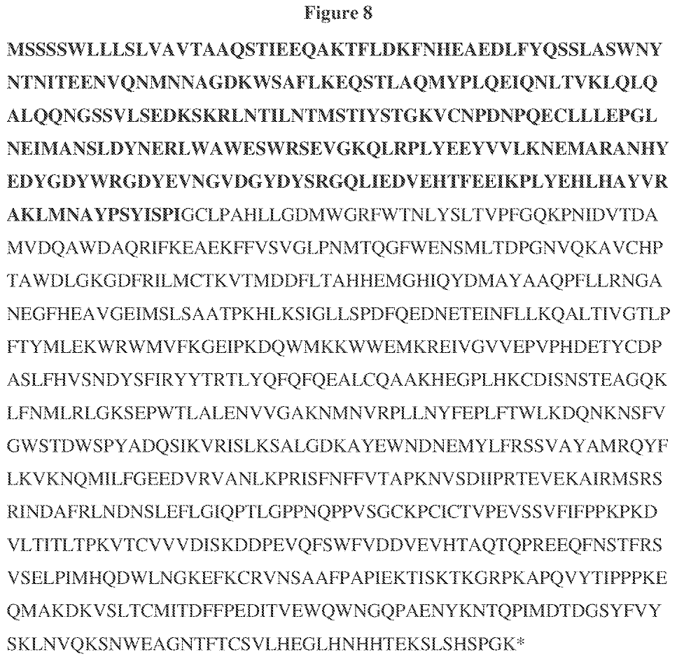

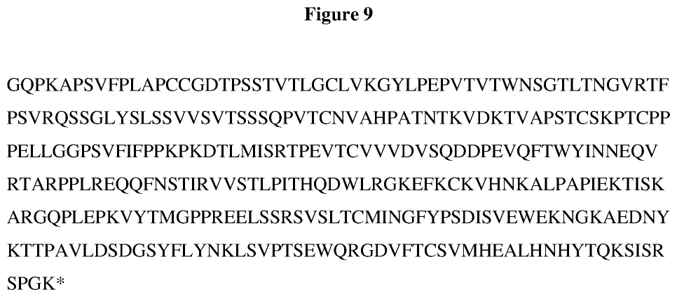

The ACE2 receptor gene was amplified from the A549 cell line. The mouse Fc region of IgG1 is from a mouse B cell. Two gene fragments will be joined by overlap extension polymerase chain reaction and cloned into pLenti6 V5 expression plasmid (Invitrogen) for expression in HEK293T cells. The sequence of Chimeric Protein 4 is shown in FIG. 8. Bold indicates the ACE2 receptor protein portion.

What is claimed is:

1. The method for determining if a human possesses at least one type of neutralizing antibody against SARS-Cov-2 as claimed in claim 2, wherein steps a)-f) are performed sequentially in order as set forth.

2. A lateral flow assay method for determining if a human possesses at least one type of neutralizing antibody against SARS-Cov-2, comprising steps of:
a) incubating a body fluid with at least one chimeric protein comprising a receptor binding domain of a variant of SARS-Cov-2 or a receptor binding domain of a variant of SARS-Cov-2 conjugated with horseradish peroxidase for a period of time to form Mixture 1;
b) adding an additional chimeric protein comprising ACE2 fused with a mouse immunoglobulin G1 fragment to the Mixture 1 formed in step (a) and incubating for a period of time to form Mixture 2;
c) contacting the Mixture 2 with anti-mIgG;
d) visualizing whether the anti-mIgG produces a visible signal emitted by the horseradish peroxidase or a visualizable tag attached to the at least one chimeric protein comprising the receptor binding domain of the variant of SARS-Cov 2; and
f) if the anti-mIgG produces a visible signal, concluding that the body fluid does not contain at least one type of neutralizing antibody against SARS-Cov-2.

3. (canceled)

4. The method of claim 2, further comprising a steps of adding rabbit IgG attached to a visualizable tag to the Mixture 2 prior to step (c), and contacting the Mixture 2 from step (d) with anti-rIgG and subsequently determining the anti-rIgG produces a signal in order to indicate proper operation of the method and use of a sufficient amount of sample in the method.

5. The method of claim 2, in which steps (c) and (d) are carried out sequentially with step (c) performed prior to step (d).

6. The method of claim 2, in which steps (c) and (d) are carried out simultaneously.

7. The method of claim 2, wherein the body fluid is whole blood or serum; the period of time in step (a) is 10-15 minutes; the period of time in step (b) is 10-15 minutes; and the receptor binding domain of the chimeric protein is conjugated to horseradish peroxidase.

8. The method of claim 2, wherein the body fluid is whole blood or serum; and the period of time in step (a) is 10-15 minutes; the period of time in step (b) is 10-15 minutes.

9. The method of claim 4, wherein the rabbit IgG has SEQ ID NO: 5 and the visualizable tag attached to the rabbit IgG is nanogold particles.

10. A kit for use in a method for determining if a human has at least one type of neutralizing antibody against SARS-Cov-2 comprising:
a. a container containing at least one chimeric protein comprising a receptor binding domain of a variant of SARS-Cov-2 or a receptor binding domain of a variant of SARS-Cov-2 conjugated with horseradish peroxidase;
b. an additional chimeric protein comprising ACE2 fused with a mouse immunoglobulin G1 fragment;
c. a unit comprising the following elements laid out in the following successive order:
(i) a sample pad onto which the at least one chimeric protein comprising a receptor binding domain of a variant of SARS-Cov-2 or a receptor binding domain of a variant of SARS-Cov-2 conjugated with horseradish peroxidase, and a body fluid is loaded;
(ii) a conjugate pad onto which the additional chimeric protein comprising ACE2 fused with a mouse immunoglobulin G1 fragment is loaded;
(iii) a test pad in contact with the sample pad and including a test line in which anti-mIgG is immobilized;
(iv) an optional absorption pad in contact with the test pad to facilitate flow of liquid from the sample pad through the test pad; and
(v) an optional backing card onto which elements (i)-(iv) are mounted; and
d. instructions for use of the kit for determining if a human has at least one type of neutralizing antibody against SARS-Cov-2.

11. The kit of claim 10, further comprising rabbit IgG attached to a visualizable tag for loading onto the test pad, and an anti-rIgG control line on the test pad downstream of the test line.

12. The kit of claim 10, wherein the receptor binding domain of the at least one chimeric protein is attached to gold nanoparticles and the visualizable tag attached to the rabbit IgG is gold nanoparticles.

13-15. (canceled)

16. An ELISA method for determining if a human possesses at least one type of neutralizing antibody against SARS-Cov-2, comprising steps of:
a) incubating a body fluid with at least one chimeric protein comprising a receptor binding domain of a variant of SARS-Cov-2 conjugated with horseradish peroxidase for a period of time to form Mixture 1;
b) adding Mixture 1 to a plate coated with an additional chimeric protein comprising ACE2 fused with a mouse immunoglobulin G1 fragment;
c) capturing unbound chimeric protein comprising a receptor binding domain of a variant of SARS-Cov-2 conjugated with horseradish peroxidase on the plate, and removing chimeric protein comprising a receptor binding domain of a variant of SARS-Cov-2 conjugated with horseradish peroxidase that is bound to neutralizing antibody by washing with phosphate buffer; and
d) visualizing horseradish peroxidase activity by adding 10-Acetyl-3,7-dihyroxyphenoxazine;
e) if no signal indicative of horseradish peroxidase activity is visible, concluding that the body fluid contains at least one type of neutralizing antibody against SARS-Cov 2; and
f) if a signal indicative of horseradish peroxidase activity is visible, concluding that the body fluid does not contain at least one type of neutralizing antibody against SARS-Cov 2.

17. The method of claim 2, wherein the receptor binding domain of the at least one chimeric protein is selected from the group consisting of SEQ ID NO: 1, SEQ ID NO: 2 and SEQ ID NO: 3.

18. The method of claim 17, wherein the comprising ACE2 fused with a mouse immuoglobulin G1 fragment has SEQ ID NO: 4.

19. The method of claim 2, wherein the receptor binding domain of the at least one chimeric protein is conjugated to gold nanoparticles.

20. The kit of claim 10, wherein the receptor binding domain of the at least one chimeric protein is selected from the group consisting of SEQ ID NO: 1, SEQ ID NO: 2 and SEQ ID NO: 3.

21. The kit of claim 20, wherein the additional chimeric protein comprising ACE2 fused with a mouse immunoglobulin G1 fragment has SEQ ID NO: 4.

22. The kit of claim 11, wherein the rabbit IgG has SEQ ID NO: 5.

23. The ELISA method of claim 16, wherein the receptor binding domain of the at least one chimeric protein is selected from the group consisting of SEQ ID NO: 1, SEQ ID NO: 2 and SEQ ID NO: 3 and the additional chimeric protein comprising ACE2 fused with a mouse immunoglobulin G1 fragment has SEQ ID NO: 4.


SEQUENCE LISTING (5 sequences)

SEQ ID NO: 1 - protein, 481 aa, SARS-COV-2
  RBD of spike protein
MFVFLVLLPLVSSQRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFMSKGEELFTGVVPILVELDGDVNGHKFSVSGEGEGDATYGKLTLKFICTTGKLPVPWPTLVTTFSYGVQCFSRYPDHMKRHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSALSKDPNEKRDHMVLLEFVTAAGITHGMDELYKHHHHHH

SEQ ID NO: 2 - protein, 555 aa, SARS-COV-2
  RBD of spike protein
MFVFLVLLPLVSSQRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFMQLTPTFYDNSCPNVSNIVRDIIVNELRSDPRIAASILRLHFHDCFVNGCDASILLDNTTSFRTEKDAFGNANSARGFSVIDRMKAAVESACPGTVSCADLLTIAAQQSVTLAGGPSWRVPLGRRDSLQAFLDLANANLPAPFFTLPQLKDSFRNVGLNRSSDLVALSGGHTFGKSQCRFIMDRLYNFSNTGLPDPTLNTTYLQTLRGLCPLNGNLSALVDFDLRTPTIFDNKYYVNLEEQKGLIQSDQELFSSPDATDTIPLVRSFANSTQTFFNAFVEAMDRMGNITPLTGTQGQIRRNCRVVNSNSDLHHHHHHH

SEQ ID NO: 3 - protein, 555 aa, SARS-COV-2
  RBD of spike protein
MFVFLVLLPLVSSQRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFMQLTPTFYDNSCPNVSNIVRDIIVNELRSDPRIAASILRLHFHDCFVNGCDASILLDNTTSFRTEKDAFGNANSARGFSVIDRMKAAVESACPGTVSCADLLTIAAQQSVTLAGGPSWRVPLGRRDSLQAFLDLANANLPAPFFTLPQLKDSFRNVGLNRSSDLVALSGGHTFGKSQCRFIMDRLYNFSNTGLPDPTLNTTYLQTLRGLCPLNGNLSALVDFDLRTPTIFDNKYYVNLEEQKGLIQSDQELFSSPDATDTIPLVRSFANSTQTFFNAFVEAMDRMGNITPLTGTQGQIRRNCRVVNSNSDLHHHHHHH

SEQ ID NO: 4 - protein, 962 aa, Artificial Sequence
  ACE2-mIgG1Fc or Chimeric Protein 4
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 5 - protein, 323 aa, Rabbit
  IgG sequence
GQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPITHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYNKLSVPTSEWQRGDVFTCSVMHEALHNHYTQKSISRSPGK